(19) United States
(12) Patent Application Publication
(10) Pub. No.: US 2011/0130704 A1
(43) Pub. Date: Jun. 2, 2011

(54) COMBINATION OF A LIGHT RAY AND A LIPASE-BIOCONVERTIBLE COMPOUND FOR IMPROVING SKIN AND/OR HAIR APPEARANCE

(75) Inventors: Francine Baldo, Sceaux (FR); Quang Lan Nguyen, Antony (FR); Dang-Man Pham, Sucy-en-brie (FR)

(73) Assignee: L'OREAL, Paris (FR)

(21) Appl. No.: 12/919,371

(22) PCT Filed: Feb. 25, 2009
(86) PCT No.: PCT/FR2009/050303

§ 371 Date: Dec. 3, 2010

Related U.S. Application Data
(60) Provisional application No. 61/032,976, filed on Mar. 2, 2008.

(30) Foreign Application Priority Data
Feb. 25, 2008 (FR) 0851178

(51) Int. Cl. A61M 37/00; A61K 31/22; A61K 31/612; A61K 31/34; A61K 31/23; A61P 29/00; A61P 31/00; A61P 17/10; A61K 8/37; A61K 8/49; A61Q 19/00

(52) U.S. Cl. 604/20; 514/546; 514/164; 514/474; 514/552

(57) ABSTRACT

The present invention relates to a cosmetic method intended in particular to improve the appearance of the skin and/or hair comprising the simultaneous and/or sequential administration:
a) of at least one compound bioconvertible by lipase; and
b) of at least one light radiation exhibiting at least one predominant wavelength which activates lipase.

In particular, the said light radiation according to the invention exhibits (emits) at least one predominant wavelength which activates lipase ranging from 400 to 510 nm, preferably from 430 to 500 nm, and is preferably used at a dose ranging from 0.01 to 200 J/cm², preferably from 0.1 to 30 J/cm², more preferably from 1 to 30 J/cm², indeed even from 5 to 30 J/cm².

The invention also relates to a composition comprising at least one compound bioconvertible by lipase and at least one compound which emits and/or filters, in particular under exposure to light, a light radiation exhibiting at least one predominant wavelength which activates lipase, as well as to a kit comprising at least one composition comprising at least one compound bioconvertible by lipase as well as a device and/or a compound which emits/filters a light radiation exhibiting at least one predominant wavelength which activates lipase.

The present invention relates to the field of the care of the skin and/or hair and in particular of the improvement in the appearance of the skin and/or hair by light radiation.

The term “skin” is understood to mean the skin of the face and/or of the body and also the scalp.

In particular, for the face, the invention preferably relates to the area of the forehead and of the nose and, for the body, preferably the area of the back.

The present invention relates in particular to a cosmetic method intended in particular to improve the appearance of the skin and/or hair comprising the simultaneous and/or sequential administration:
of at least one compound bioconvertible by lipase; and
of at least one light radiation exhibiting at least one predominant wavelength which activates lipase.
   The cosmetic method according to the present invention comprises, in particular, the simultaneous and/or sequential administration, to a subject, in particular to a subject having healthy skin:
of at least one compound bioconvertible by lipase; and
of at least one light radiation exhibiting at least one predominant wavelength which activates the lipase.

The term “healthy skin” is understood to mean skin not exhibiting a lesion and/or an infection.

In particular, the said light radiation according to the invention exhibits (emits) at least one predominant wavelength which activates lipase ranging from 400 to 510 nm, preferably from 430 to 500 nm, and is preferably used at a dose ranging from 0.01 to 200 J/cm², preferably from 0.1 to 30 J/cm², more preferably from 1 to 30 J/cm², indeed even from 5 to 30 J/cm².

The invention also relates to a composition comprising at least one compound bioconvertible by lipase and at least one compound which emits and/or filters, in particular under exposure to light, a light radiation exhibiting at least one predominant wavelength which activates lipase, as well as to a kit comprising at least one composition comprising at least one compound bioconvertible by lipase and a device and/or a compound which emits/filters a light radiation exhibiting at least one predominant wavelength which activates lipase.

The retaining of a young appearance and/or healthy skin and/or hair is resulting in an unceasing search for novel compounds and/or novel noninvasive treatment methods which make it possible to maintain or to improve the appearance of the skin and/or hair.

There is a search in particular to prevent and/or alleviate visible and/or tactile irregularities of the skin and/or hair.

In particular, there is a search to prevent and/or alleviate skin imperfections of greasy skin or skin with a tendency towards acne and/or greasy scalps.

The term “skin imperfections of greasy skin” is understood to mean in particular, according to the invention, aesthetic disorders, such as glistening skin, poorer hold of the makeup, a thick skin texture generally associated with a lack of desquamation, skin having follicular orifices which are dilated (increased visibility of the pores) or filled with minute horny spicules, indeed even by comedones or blackheads (resulting, however, more from a phenomenon of retention than from an increase in the excretion), or a loss in radiance and/or in transparency of the skin.

The appearance and/or the visibility of the pores in particular is especially increased in people exhibiting dilated pores and/or an excess of sebum, in particular in persons having Asian or Caucasian skin.

The shininess of the skin is a problem affecting more particularly adolescents but which can also be displayed during adulthood under the effect in particular of hyperproduction of androgens. A shiny or greasy skin is generally a hyperseborrhoeic skin characterized by an excessive secretion and an excessive excretion of sebum, generally resulting in a level of sebum of greater than 200 μg/cm², measured on the forehead.

Sebum is the natural product from the sebaceous gland, which constitutes an appendix of the pilosebaceous unit. It is essentially a more or less complex mixture of lipids. Conventionally, the sebaceous gland produces squalene, triglycerides, aliphatic waxes, cholesterol waxes and possibly free cholesterol (Stewart, M. E., Semin Dermatol, 11, 100-105 (1992)). A variable portion of the triglycerides formed is converted to free fatty acids under the action of bacterial lipases.

The sebocyte is the relevant cell of the sebaceous gland. The production of sebum is associated with a terminal differentiation programme of this cell. During this differentiation, the metabolic activity of the sebocyte is essentially centred on the biosynthesis of the lipids (lipogenesis) and more specifically on fatty acid neosynthesis.

While sebum is normally a natural moisturizer for the epidermis, the excess production of sebum (hyperseborrhoea) can have unpleasant effects aesthetically, such as glistening skin, poorer hold of the makeup, increased visibility of the pores, the formation of comedones or blackheads, or a loss in radiance and/or in transparency.

Sebum can in addition be a medium favourable to colonization by bacteria of P. acnes type, implicated in acne.

It can also promote colonization by bacteria of P. ovale type, implicated in the formation of dandruff on the hair and/or in inflammatory phenomena on the scalp.

An excess of sebum and the peroxidation of the lipids of the sebum can also detrimentally affect the biology of the hair and thus slow down its growth or promote its loss, its greying, a decrease in the diameter of the hairs or a decrease in the vigour of the hairs.

The skin imperfections of greasy skin can be more or less visible according to the phototype of the subject; in particular, they are more visible on the skin of subjects with a high phototype, that is to say skin of subjects with phototype III to VI, defined according to the Fitzpatrick classification, which is based on the reactivity of the skin to the effects of solar radiation:
I Always burns, never tans
II Always burns, tans very little
III Burns moderately, tans gradually
IV Burns slightly, tans very easily
V Burns rarely, tans deeply
VI Never burns, highly pigmented.

The visibility of the pores also is accentuated in people having aged skin, in particular photoaged skin, and also particularly in people having Asian or Caucasian skin.

The greasy skin of the face of men, subjected to daily shaving, is in addition particularly prone to skin lesions.

In order to combat excess production of sebum or hyperseborrhoea, various compounds have thus been proposed in the prior art which, by topical application on the skin, are capable of reducing lipogenesis in the sebocytes and of consequently limiting the production of sebum.

Mention may be made, for example, of the following compounds:
powders which make it possible to absorb sebum and thus to render the skin matt;
α- or β-hydroxy acids which, due to their keratolytic effect, promote the expulsion of horny plugs;
the astringent active principles which make it possible to combat the dilation of sebaceous follicles;
exfoliating active principles which make it possible to reduce the thickness of the skin; and
active principles which act on the biochemical mechanisms, in particular enzymes, involved in the production of sebum.

However, the need remains to have available novel compounds and/or novel noninvasive methods for the treatment of the skin and/or hair which make it possible to improve the appearance of the skin and/or hair, in particular of greasy skin and/or skin with a tendency towards acne.

The difficulty is to find compounds and/or systems which are capable of acting specifically on the sebaceous follicles (thus preferably lipophilic for better bioavailability) and which are capable of reducing the production of sebum (and not increasing it, as is the case with lipophilic active principles, which are prosebogenic by definition) and/or killing bacteria (for example P. acnes or P. ovale).

The Applicant Company has discovered, surprisingly, that it is possible to respond to this problem by combining lipophilic active principles bioconvertible by lipase with a light radiation exhibiting at least one predominant wavelength which activates lipase, in particular at the sebaceous glands.

Specifically, the Applicant Company has been able to show, for example, that a light radiation exhibiting a predominant wavelength ranging from 400 to 510 nm (emission spectrum of blue light) makes it possible to increase the bioconversion of ethyl lactate to ethanol, which is bactericidal with regard to P. acnes, and to lactic acid, the desquamating activity of which is beneficial for regulating greasy skin. This is applicable to other compounds bioconvertible by lipase, such as retinol palmitate, ascorbyl palmitate and many other compounds described below.

Propionibacterium acnes (P. acnes) is an anaerobic bacterium predominant in the sebaceous region. The lipase produced by P. acnes has a molecular weight of 46.77 Da and its optimum pH is between 5 and 6 (Ingham et al., Characterization of lipase from Propionibacterium acnes, J. Gen. Microbiol., 124(2), 393-401, 1981).
This enzyme shows no positional preference of fatty acids.
It participates in the anchoring of P. acnes in the infundibulum by release of oleic acid from triolein, which is necessary for the nutrition of the bacterium and for its adhesion (Zouboulis C C et al., What is the pathogenesis of acne?, Experimental Dermatology 2005, 14, 143-152).
This presence of P. acnes in large amounts in the follicle is one of the factors causing the inflammation in acne (Mark D et al., Acne: inflammation, J. Clin. Dermatol., 2004, 03, 0006).

Hence the interest, in the case of greasy skin, of being able to divert the mechanism of a lipase towards the formation of active principles in situ capable of regulating the production of sebum, reducing the imperfections of greasy skin and/or inhibiting P. acnes and thus preventing and/or reducing the resulting inflammation present in acneic lesions.

The use was known, from Application WO 03/17824, of a composition comprising a chromophore in combination with light radiation for treating disorders relating to the activity of the sebaceous glands, in particular acne.

However, to the knowledge of the Applicant Company, the stimulating effect on lipase of a light radiation, in particular of a light radiation exhibiting at least one predominant wavelength ranging from 400 to 510 nm (emission spectrum of blue light), had never been described until now, nor had the specific combination of such a light radiation with a compound bioconvertible by lipase, in particular a compound bioconvertible to give an agent active in situ for the problems of greasy skin and/or acne.

The combination according to the invention of a light radiation exhibiting at least one predominant wavelength which activates lipase and of a compound bioconvertible by lipase thus makes it possible to convey the lipophilic bioconvertible compounds to the bottom of the sebaceous follicles for release, after hydrolysis by the lipase, at the site of production of the sebum and of possible colonization by bacteria of type P. acnes or P. ovale, of compounds (for example alcohols or acids) with properties in combating greasy skin or acne.

This combination also makes it possible to reduce the duration and/or the intensity of the light source administered, while retaining a satisfactory effectiveness.

The invention thus relates to the combination of at least one compound bioconvertible by lipase with at least one light radiation exhibiting at least one predominant wavelength which activates lipase, in particular a light radiation exhibiting at least one predominant wavelength ranging from 400 to 510 nm (emission spectrum of blue light), in a cosmetic treatment method, a composition, a kit or a therapeutic use which are intended in particular to improve the appearance of the skin and/or hair.

The compound bioconvertible by lipase can be formulated in a composition for administration topically and/or orally, preferably topically.

The light radiation exhibiting at least one predominant wavelength which activates the lipase can be in the form of a device or, according to an alternative, in the form of a compound capable of emitting and/or filtering, in particular under exposure to light, at least one predominant wavelength which activates lipase. In the latter case, the said compound which emits and/or filters the said light radiation which activates lipase can be formulated in the same composition as the compound bioconvertible by lipase or in a separate composition.

A subject-matter of the invention is thus a cosmetic method intended in particular to improve the appearance of the skin and/or hair comprising the simultaneous and/or sequential administration:
a) of at least one compound bioconvertible by lipase; and
b) of at least one light radiation exhibiting at least one predominant wavelength which activates lipase.

Advantageously, all the cosmetic methods according to the present invention are employed on subjects having healthy skin.

The term “light radiation exhibiting at least one predominant wavelength” is understood to mean, according to the invention, a light radiation distinct from white light comprising all the wavelengths of the spectrum.

This is because it is known that each wavelength has a specific target in the cells of the skin and/or hair, known as chromophore or photoacceptor.

Specifically, the Applicant Company has shown that some wavelengths are capable of activating lipase, whereas others are without effect.

Preferably, the light radiation exhibits at least one predominant wavelength ranging from 400 to 510 nm, preferably from 420 to 500 nm and more preferably from 430 to 470 nm (emission spectrum of blue light). Use will in particular be made of a light radiation, the spectrum of which has an optimum peak in the vicinity of 450 nm. More particularly, the optimum peak of the light radiation used will be in the vicinity of 450 nm±50 nm, preferably in the vicinity of 450 nm±30 nm and entirely preferably in the vicinity of 450 nm±20 nm.

According to a preferred form, the light radiation exhibiting at least one predominant wavelength which activates lipase is applied to the skin and/or hair, in particular to the areas of skin and/or hair treated with a composition comprising at least one compound bioconvertible by lipase, at a dose ranging from 0.01 to 200 J/cm², preferably from 0.1 to 30 J/cm².

The term “sequential” is understood to mean a successive (immediate) or delayed administration. In the case of a sequential administration, and advantageously, the compound bioconvertible by lipase is administered before the administration of the light radiation exhibiting at least one predominant wavelength which activates lipase but the said compound bioconvertible by lipase can also and complementarily be administered after a compound bioconvertible by lipase+light radiation exhibiting at least one predominant wavelength which activates lipase session.

According to a specific form, the light radiation exhibiting at least one predominant wavelength which activates lipase is a compound which emits and/or filters, in particular under exposure to light or UV radiation, the said light radiation which activates lipase; the said compound can be formulated in the composition comprising the said compound bioconvertible by lipase or else in another topical composition intended to be applied separately in time (before or after).

According to another form, use is made of a patch, in particular an iontophoretic patch, impregnated with at least one compound bioconvertible by lipase and provided with diodes which emit the said light radiation which activates lipase.

The use of the method according to the invention can additionally comprise a stage, prior to or concomitant with the stages of the method according to the invention, targeted at improving the penetration of the compound bioconvertible by lipase, for example while cooling the skin, by iontophoresis or by an occlusive system.

It is also possible to promote the penetration of the compound bioconvertible by lipase by carrying out a chemical or mechanical peeling beforehand on the area to be treated.

Such a combination according to the invention has in particular the effect of regulating the production of sebum (taken advantage of in treating in particular greasy skin) and/or of killing the bacteria in the sebaceous follicle (taken advantage of in treating in particular acne).

This combination thus makes it possible to maintain and/or improve the appearance of the skin and/or hair, in particular of greasy skin and/or of greasy scalps.

Furthermore, it is known that an excess of sebum and the oxidation of the latter influences the physiology of the hair and can in particular contribute to hair loss, the slowing down in its growth, its greying, the reduction in the diameter of the hairs and the reduction in the vigour of the hairs.

The method according to the invention is thus targeted in particular at alleviating visible or tactile irregularities of the surface of the skin and/or scalp, in particular of greasy skin.

In particular, the method according to the invention is targeted at alleviating skin imperfections of greasy skin, in particular at alleviating the shininess of the skin, alleviating the visibility of the pores, improving the radiance and/or the transparency of the skin, improving the hold of the makeup, improving the texture of the skin and preventing and/or reducing the formation of comedones.

Another object is targeted at a method intended to prevent and/or combat hair loss, the slowing down in its growth, its greying, the reduction in the diameter of the hairs and/or the reduction in the vigour of the hairs.

The invention also relates to the combination (i) of at least one compound bioconvertible by lipase and (ii) of at least one light radiation exhibiting at least one predominant wavelength which activates lipase for regulating the production of sebum and/or killing (destroying) bacteria, in particular in the sebaceous follicles.

The invention also relates to the use of the combination (i) of at least one compound bioconvertible by lipase and (ii) of at least one light radiation exhibiting at least one predominant wavelength which activates lipase in the preparation of a composition intended to regulate the production of sebum and/or to kill (destroy) bacteria, in particular in the sebaceous follicles.

The compound bioconvertible by lipase is in particular capable of releasing, after hydrolysis in situ by the lipase in the sebaceous follicle, at least one agent active with regard to acne having antibacterial and/or anti-inflammatory activity. Examples of such compounds bioconvertible by lipase will be described below.

In particular, the combination according to the invention can be used to prevent and/or reduce red skin blotches of inflammatory origin or to prevent and/or treat inflammatory lesions, in particular acneic lesions.

The combination according to the invention can also be used to prepare a composition intended to reduce red skin blotches of inflammatory origin or to prevent and/or treat inflammatory lesions, in particular acneic lesions.

According to another form, the combination according to the invention can be used to prevent and/or treat the formation of dandruff and/or inflammatory phenomena of the scalp.

In particular, the areas to be treated according to the invention concern:
on the face, in particular the T area (forehead, nose, cheeks and chin), in particular the area of the nose and forehead;
on the body, the area of the back and more particularly the top of the back;
the scalp.

According to a specific form, the cosmetic method and/or the therapeutic use according to the invention can be employed on any type of greasy skin and in particular:
masculine greasy skin, in particular subjected to daily shaving, particularly prone to skin lesions;
aged greasy skin, in particular photoaged greasy skin;
greasy skin of high phototype (III to VI), where the skin imperfections are generally more visible;
greasy skin of ethnic type, in particular Asian skin or Caucasian skin, where the visibility of the pores is generally accentuated;
greasy skin with a tendency towards acne, in particular in adolescence.

### Compounds Bioconvertible by Lipase

The compound bioconvertible by lipase can be formulated in a composition intended for oral or topical administration.

According to a preferred form of the invention, the compound bioconvertible by lipase is formulated in a composition intended for topical administration to the skin or hair.

The term “compounds bioconvertible by lipase” which can be used according to the invention is understood to mean compounds capable of being hydrolysed by lipase in order to release in particular agents beneficial to the skin.

According to a specific form, the compounds bioconvertible by lipase used according to the invention are capable, when they are hydrolysed in situ by the lipase in the sebaceous follicle, of releasing at least one agent (compound) active with regard to greasy skin or with regard to acne.

The term “agent active with regard to greasy skin” is understood in particular to mean according to the invention a compound having a desquamating (keratolytic), seboregulating (reduces the production of sebum) and/or antioxidant (reduces in particular the peroxidation of lipids capable of contributing to the formation of comedones) activity.

The antioxidant activity in addition makes it possible, by reducing the formation of comedones (plugs which reinforce the anaerobic side favourable to the development of P. acnes), to prevent the development of P. acnes.

The term “agent active with regard to acne” is understood in particular to mean according to the invention a compound having an antibacterial and/or anti-inflammatory activity.

According to a specific form, the said agent active with regard to greasy skin or with regard to acne comprises at least one —OH or —COOH group.

According to a specific form (cosmetic treatment method), the compound bioconvertible by lipase is a compound capable of releasing, after hydrolysis by the lipase in situ, at least one agent active with regard to greasy skin having a desquamating, seboregulating and/or antioxidant activity.

Preferably, the compound bioconvertible by lipase is a lipophilic compound, in particular a compound having at least one ester functional group, intended in particular to penetrate at the level of the sebaceous follicle.

In particular, the compound bioconvertible by lipase used according to the invention is a compound comprising one or more ester functional groups, preferably mono- and diester functional groups, possessing a saturated or unsaturated and linear, cyclic or branched chain having from 2 to 50 carbon atoms, preferably from 6 to 50 carbon atoms, and optionally comprising one or more substituents.

Thus, the lipase activated by the light radiation according to the invention and preferably a light radiation exhibiting at least one predominant wavelength ranging from 400 to 510 nm, preferably from 420 to 470 nm, preferably from 420 to 500 nm and more preferably from 430 to 470 nm hydrolyses the said bioconvertible compound preferably comprising at least one ester functional group in order to release at least one agent (compound) active with regard to greasy skin (i) and/or acne (ii) having at least one activity of:
seboregulating, desquamating and/or antioxidant type (intended in particular to prevent the peroxidation of lipids)-(i);
antibacterial and/or anti-inflammatory type-(ii).
   According to a specific form, the said agent (compound) released after hydrolysis which is active with regard to greasy skin or acne comprises at least one —OH or —COOH group.

By way of example, the compound bioconvertible by lipase can release, after hydrolysis in situ by the lipase in the sebaceous follicle, at least:
a) one compound comprising at least one —OH or —COOH group having an activity in combating greasy skin or acne;
b) one compound comprising at least one —OH or —COOH group and one agent active with regard to greasy skin and/or acne,
c) one compound comprising at least one —OH group and one compound comprising at least one —COOH group,
   it being possible for the said compound(s) comprising at least one —OH or —COOH group in the alternatives a) and b) themselves to have an activity of seboregulating, keratolytic (desquamating), antioxidant (intended in particular to prevent the peroxidation of lipids), antibacterial and/or anti-inflammatory activity.

Apart from the compound having an activity in combating greasy skin/acne, the bioconvertible compound can also release, by hydrolysis in situ at the sebaceous follicle, other compounds beneficial to the skin having, for example, a hydrating, antiageing or softening activity on the skin.

A person skilled in the art will choose the bioconvertible compound to be administered according to the desired effect (combating greasy skin or acne, and optionally in combination a supplementary hydrating, antiageing or softening activity on the skin).

Mention may in particular be made, as compound released after hydrolysis by lipase, comprising at least one —COOH group and having an activity in combating greasy skin or acne, of compounds comprising at least one —COOH group possessing a saturated or unsaturated and linear, cyclic or branched chain having from 2 to 50 carbon atoms, preferably from 6 to 50 carbon atoms, and optionally comprising one or more substituents.

Mention may be made, for example, of α-hydroxy acids, in particular glycolic acid or lactic acid, β-hydroxy acids, in particular salicylic acid, fatty acids, such as palmitic acid, caprylic/capric acid, linoleic acid, benzoic acid, retinoic acid, glycyrrhetinic (C₃₀) acid, sorbic acid, asiatic (C₄₈) acid, azelaic acid, ursolic acid, 10-hydroxy-2-decanoic acid, 4-(2-hydroxy-ethyl)piperazine-1-ethanesulphonic acid (HEPES), phthalimidoperoxycaproic acid, pyrrolidonecarboxylic acid, oleanolic acid, betulinic acid, isovaleric acid, butyric acid, acetic acid, octanoic acid, propionic acid, carboxymethylcysteine and their derivatives.

The hydrogen atoms of the carbon-comprising chain of these acids can, in addition, be substituted by halogens or halogenated, alkyl, acyl, acyloxyl, alkoxycarbonyl or alkoxyl radicals having from 2 to 18 carbon atoms.

Mention may in particular be made, as compound released by hydrolysis by lipase, comprising at least one —OH group and having an activity in combating greasy skin or acne, of substituted or unsubstituted, mono-, di- or trihydroxylated and linear, cyclic or branched C₂-C₂₂ compounds.

Mention may be made, for example, of ethanol, glycerol, ethyl glycerol, pentylene glycol, caprylyl glycol, resveratrol (C₁₄), retinol (C₂₀), 3,7,11-trimethyl-2,6,10-dodecatrienol (or farnesol), oleyl alcohol, octanol or 2-ethylhexanol, palmityl alcohol, chimyl alcohol, batyl alcohol, capryl alcohol, panthenol, tocopherol, phytosphingosine, octyldodecanol, ascorbic acid and terpene alcohols (for example totarol, geraniol, and the like).

Mention may be made, as other compounds released having an antibacterial and/or anti-inflammatory activity, of hydrocortisone or betamethasone.

A person skilled in the art will thus know to choose, according to the effect desired, the compounds released which are suitable for a cosmetic use (desquamating, seboregulating and/or antioxidant activity) or a therapeutic use (antibacterial and/or anti-inflammatory activity) and consequently will know to choose a corresponding ester bioconvertible in situ by lipase.

According to a first embodiment (cosmetic treatment method, in particular for greasy skin), the compound bioconvertible by lipase is a compound, preferably a compound exhibiting at least one ester functional group, capable of releasing, after hydrolysis in situ by the lipase in the sebaceous follicle, at least one agent active with regard to greasy skin, such as a compound having a desquamating, seboregulating and/or antioxidant activity.

According to a specific form, the said agent (compound) active with regard to greasy skin comprises at least one —OH or —COOH group.

In particular, the compound bioconvertible by lipase releases, after hydrolysis in situ by the lipase in the sebaceous follicle, at least one saturated or unsaturated and linear, branched or cyclic compound having from 2 to 50 carbon atoms, preferably from 6 to 50 carbon atoms, comprising at least one —COOH group and having a desquamating, seboregulating and/or antioxidant activity.

The compound bioconvertible by lipase can also release, after hydrolysis in situ by the lipase in the sebaceous follicle, at least one substituted or unsubstituted, mono-, di- or trihydroxylated and linear, cyclic or branched C₂-C₂₂ compound having a desquamating and/or seboregulating and/or antioxidant activity.

Use may in particular be made, as compound bioconvertible by lipase capable of releasing, by hydrolysis in situ in the sebaceous follicle, compounds having a high desquamating, seboregulating and/or antioxidant valency, of a compound chosen from esters of:
α-hydroxy acids, in particular glycolic acid or lactic acid, β-hydroxy acids, in particular salicylic acid (at a low concentration in order to express its desquamating valency), 10-hydroxy-2-decanoic acid, 4-(2-hydroxy-ethyl)piperazine-1-ethanesulphonic acid (HEPES), phthalimidoperoxycaproic acid or carboxymethylcysteine,
glycerol, ethyl glycerol, pentyleneglycol, caprylyl glycol, resveratrol (C₁₄), retinol (C₂₀), octanol or 2-ethylhexanol, palmityl alcohol, tocopherol, phytosphingosine, octyldodecanol or ascorbic acid, and their mixtures.
   For a cosmetic use, the esters will preferably be chosen so that they do not additionally release a compound having an antibacterial and/or anti-inflammatory valency preferably reserved for a therapeutic use.

According to another embodiment (therapeutic use, in particular for treating acne), the compound bioconvertible by lipase is a compound, preferably a compound exhibiting at least one ester functional group, capable of releasing, after hydrolysis in situ by the lipase in the sebaceous follicle, at least one agent active with regard to acne, such as a compound having an antibacterial and/or anti-inflammatory activity.

According to a specific form, the said agent (compound) active with regard to acne comprises at least one —OH or —COOH group.

In particular, the compound bioconvertible by lipase releases, after hydrolysis by the lipase in situ in the sebaceous follicle, at least one saturated or unsaturated and linear, branched or cyclic compound having from 2 to 50 carbon atoms, preferably from 6 to 50 carbon atoms, comprising at least one —COOH group and having an antibacterial and/or anti-inflammatory activity.

The compound bioconvertible by lipase can also release, after hydrolysis in situ by the lipase in the sebaceous follicle, at least one substituted or unsubstituted, mono-, di- or trihydroxylated and linear, cyclic or branched C₂-C₂₂ compound comprising at least one —OH group and having an antibacterial and/or anti-inflammatory activity.

Use may in particular be made, as compound bioconvertible by lipase capable of releasing, by hydrolysis in situ in the sebaceous follicle, compounds having a high antibacterial and/or anti-inflammatory valency, of a compound chosen from esters of:
salicyclic acid (at a high concentration in order to express its anti-inflammatory valency), caprylic/capric acid, linoleic acid, benzoic acid, retinoic acid, glycyrrhetinic acid (C₃₀), sorbic acid, asiatic acid (C₄₈), azelaic acid, ursolic acid, pyrrolidonecarboxylic acid, oleanolic acid, betulinic acid or isovaleric acid,
ethanol, 3,7,11-trimethyl-2,6,10-dodecatrienol (or farnesol), oleyl alcohol, chimyl alcohol, batyl alcohol, panthenol or capryl alcohol,
antibacterial and/or anti-inflammatory agents, in particular hydrocortisone or betamethasone.
   These esters can be used alone or as a mixture with the esters used above for the cosmetic application (greasy skin).

Mention may in particular be made, as examples of compounds bioconvertible by lipase exhibiting at least one ester functional group which can be used according to the invention, of the following compounds:
esters of salicyclic acid, such as 2-ethylhexyl salicylate(octyl salicylate), ethyl salicylate, homomenthyl salicylate or isodecyl salicylate,
AHA esters, such as esters of glycolic acid or lactic acid, for example ethyl glycolate, mono-, di- and triesters of glycolic acid, ethyl lactate, myristyl lactate, the lactate of C₁₂-C₁₅ alcohols, isostearyl lactate, menthyl lactate, lauryl lactate, isostearyl lactate, oleyl lactate or octyldodecyl lactate,
esters of 10-hydroxy-2-decanoic acid, such as ethyl 10-hydroxy-2-decanoate or pentylene glycol 10-hydroxy-2-decanoate,
esters of ascorbic acid, such as ascorbyl palmitate, ascorbyl tetraisopalmitate, ethyl ascorbate or cetyl ascorbate,
esters of alcohols having an antioxidant activity, such as tocopherol acetate,
esters of palmitic acid or palmityl alcohol, such as isopropyl palmitate, 2-ethylhexyl palmitate, retinol palmitate or isostearyl palmitate,
esters of azelaic acid, such as azelaic acid diglycinate or the diester of azelaic acid and salicylic acid,
esters of linoleic acid, such as retinol linoleate,
esters of glycyrrhetinic acid, such as stearyl glycyrrhetinate or stearyl β-glycyrrhetinate,
esters of caprylic/capric acid, such as ethyl caprate or ethyl caprylate,
esters of antibacterial and/or anti-inflammatory agents which are in particular soluble in water, such as hydrocortisone acetate, betamethasone butyrate, betamethasone valerate, hydrocortisone acetate α-propionate or pyridoxine tripalmitate,
esters of alcohols having an antibacterial/anti-inflammatory activity, such as farnesol acetate, panthenol triacetate, ethyl panthenol, esters of batyl alcohol, esters of chimyl alcohol and their mixtures,
   and their mixtures.

According to one embodiment of the present invention, the compound bioconvertible by lipase is chosen from salicylic acid esters, AHA esters, esters of 10-hydroxy-2-decanoic acid, esters of ascorbic acid, esters of alcohols having an antioxidant activity, esters of palmitic acid or palmitoyl alcohol, esters of azelaic acid, esters of linoleic acid, esters of glycyrrhetinic acid or esters of caprylic/capric acid.

According to a specific embodiment of the present invention, the compound bioconvertible by lipase is chosen from salicylic acid esters, AHA esters, esters of 10-hydroxy-2-decanoic acid, esters of ascorbic acid, esters of palmitic acid or palmitoyl alcohol, esters of azelaic acid, esters of linoleic acid, esters of glycyrrhetinic acid or esters of caprylic/capric acid.

According to another specific embodiment of the present invention, the compound bioconvertible by lipase is chosen from salicylic acid esters, AHA esters, esters of 10-hydroxy-2-decanoic acid, tocopherol acetate, esters of ascorbic acid, esters of palmitic acid or palmitoyl alcohol, esters of azelaic acid, esters of linoleic acid, esters of glycyrrhetinic acid or esters of caprylic/capric acid.

Mention may in particular be made, as preferred compounds bioconvertible by lipase, of:
ascorbyl palmitate, tocopherol acetate or retinol palmitate, suitable for the cosmetic use (for example greasy skin) according to the invention,
ethyl salicylate, ethyl lactate, esters of azelaic acid, such as azelaic acid diglycinate or the diester of azelaic acid and salicyclic acid, esters of linoleic acid, such as retinol linoleate, or esters of glycyrrhetinic acid, such as stearyl glycyrrhetinate or stearyl β-glycyrrhetinate, suitable for the therapeutic use (for example acne) according to the invention.

Use will preferably be made, to prevent and/or treat greasy skin, of ascorbyl palmitate, tocopherol acetate, retinol palmitate or their mixtures.

According to a preferred embodiment of the present invention, use will be made, in order to prevent and/or treat greasy skin, of ascorbyl palmitate.

According to another preferred embodiment of the present invention, use will be made, in order to prevent and/or treat greasy skin, of tocopherol acetate.

According to another preferred embodiment of the present invention, use will be made, in order to prevent and/or treat greasy skin, of retinol palmitate.

Use will preferably be made, for the treatment of acne, of ethyl lactate, ethyl salicylate, retinol linoleate or their mixtures.

According to a preferred embodiment of the present invention, use will be made, for the treatment of acne, of ethyl lactate.

According to another preferred embodiment of the present invention, use will be made, for the treatment of acne, of ethyl salicylate.

According to another preferred embodiment of the present invention, use will be made, for the treatment of acne, of retinol linoleate.

Mention may in particular be made, as examples of commercial references of compounds bioconvertible by lipase which can be used according to the invention, of:

| | COMMERCIAL | |
|---|---|---|
| COMPOUND | REFERENCE | SUPPLIER |
| MIXTURE OF MONO-, | GLYCERYL | GUARDIAN |
| DI- AND TRIESTER OF | GLYCOLATE | |
| GLYCOLIC ACID AND | | |
| OF POLYGLYCOLIC | | |
| ACID | | |
| MIXTURE OF CETYL | CETYLIDONE | SOLABIA |
| ESTER OF | | |
| PYRROLIDONE- | | |
| CARBOXYLIC ACID | | |
| AND OF | | |
| UNDECYLENAMIDO- | | |
| PROPYLTRIMONIUM | | |
| METHOSULPHATE | | |
| (54/26) IN WATER | | |
| PYRIDOXINE | PYRIDOXINE | DSM-ROCHE |
| TRIPALMITATE | TRIPALMITATE | VITAMINS |
| AZELAIC ACID | AZELOGLICINA | SINERGA |
| DIGLYCINATE, | | |
| POTASSIUM SALT, | | |
| AS A 30% AQUEOUS | | |
| SOLUTION, NOT | | |
| STABILIZED | | |
| FARNESYL ACETATE | UNIREDUCE R-35 | INDUCHEM AG |
| (AND) PANTHENYL | | |
| TRIACETATE (AND) | | |
| TOCOPHERYL | | |
| ACETATE | | |
| DIESTER OF AZELAIC | TU-2100 | TAMARKIN |
| ACID AND SALICYLIC | | PHARMA- |
| ACID | | CEUTICAL |
| DIESTER OF AZELAIC | RDT1004 DT 1004 | CHIMIE |
| ACID AND ETHYL | DG001 | BIOMIMETIQUE |
| LACTATE | | |
| PROTECTED VITAMIN | VITAMIN A | DSM-ROCHE |
| A LINOLEATE | LINOLEATE | VITAMINS |
| HYDROCORTISONE | MICRONIZED | VEGETADROG |
| ACETATE | HYDRO- | |
| | CORTISONE | |
| | ACETATE USP | |
| HYDROCORTISONE | HYDRO- | SAFIC-ALCAN- |
| 17-BUTYRATE | CORTISONE | SOCHIBO |
| | 17-BUTYRATE | |
| BETAMETHASONE 17- | BETAMETHASONE | OREAL |
| VALERATE | 17-VALERATE | |
| HYDROCORTISONE | HYDRO- | BEIERSDORF |
| 21-ACETATE 17-α- | CORTISONE | |
| PROPIONATE | 21-ACETATE 17-α- | |
| | PROPIONATE | |
| STEARYL | STEARYL | FACONNIER |
| GLYCYRRHETINATE | GLYCHRRHETINATE | |
| STEARYL β- | STEARYL | VINYALS |
| GLYCYRRHETINATE | GLYCYRRHETINATE | |

The said compound bioconvertible by lipase is present in the composition in an amount ranging from 0.001 to 50% by weight, preferably from 0.1 to 30% by weight, with respect to the total weight of the said composition.

Light Radiation which Activates Lipase

The term “light radiation which activates lipase” is understood to mean a light radiation used under conditions (wavelength, intensity, duration of exposure) such that it can stimulate the enzymatic activity of the lipase in the presence of a compound bioconvertible by the said enzyme and can in particular stimulate the hydrolysis of an ester.

A light radiation which activates lipase which can be used according to the invention can be selected according to an in vitro test, as described in Example 1 below, consisting in:

exposing or not exposing (control) the lipase and an ester to light radiation, for example to a power of 0.5 mW/cm²,

evaluating the effect of the said light radiation on the activity of the enzyme (hydrolysis of the ester), with respect to a control (enzyme kept sheltered from the light), and

selecting the light radiation capable of increasing the activity of the said enzyme, in particular of increasing the activity of the said enzyme by at least 10%, with respect to the nonexposed control.

Specifically, the Applicant Company has been able to show that some types of light radiation exhibiting at least one specific predominant wavelength activate lipase whereas others are without effect: in particular, it has been able to show that a radiation exhibiting a predominant wavelength corresponding to the emission spectrum of blue light (400-510 nm) applied for 90 min (dose of approximately 2.7 J/cm²) is capable of activating lipase (+33%); on the other hand, other wavelengths (for example green light or yellow-orange light) do not have an effect on this enzyme.

When light radiation is absorbed by a tissue, it delivers energy to the tissue, which itself reacts with the light radiation according to the wavelength, the intensity and the duration of exposure to the said light radiation.

The intensity of the light radiation used according to the invention preferably does not exceed 150 mW/cm² and is preferably greater than 0.01 mW/cm² (i.e. 0.01 mW/cm² to 150 mW/cm²). This intensity range ensures an effectiveness of treatment over a reasonable period of time, without causing damage to the skin tissue treated.

The duration of exposure to the light radiation will be defined, according to the intensity of the light radiation, so that the total energy delivered (dose) to the skin and/or hair is from 0.01 to 200 J/cm², preferably from 0.1 to 30 J/cm², more preferably from 1 to 30 J/cm², indeed even from 5 to 30 J/cm².

Energy (in joules)=Intensity (in watts)×Time (in seconds)

As nonlimiting example, the duration of exposure to a device emitting a light radiation according to the invention can range from 20 min to 120 min, preferably from 30 min to 120 min and more preferably still from 60 min to 90 min.

According to the surface condition of the skin and/or hair to be treated and the effect desired, it may be advantageous to carry out several applications daily of light radiation defined according to the invention in combination with a compound bioconvertible by lipase, or one application daily, indeed even one application weekly, for a period of time of one to several months.

According to a preferred form, the light radiation which activates lipase according to the invention will in particular be a light radiation exhibiting at least one predominant wavelength which activates lipase ranging from 400 to 510 nm, preferably from 420 to 500 nm and more preferably from 430 to 470 nm, with an optimum peak in the vicinity of 450 nm, and preferably used at a dose ranging from 0.01 to 200 J/cm², preferably from 0.1 to 30 J/cm², more preferably from 1 to 30 J/cm², indeed even from 5 to 30 J/cm².

According to a preferred form, light radiation exhibiting at least one predominant wavelength which activates lipase is thus applied to the skin and/or hair, in particular to the areas of skin and/or hair treated with the composition comprising at least one compound bioconvertible by lipase, at a dose ranging from 0.01 to 200 J/cm², preferably from 0.1 to 30 J/cm².

Mention may in particular be made, as “light radiation exhibiting at least one predominant wavelength which activates lipase” which can be used according to the invention, of:
a light radiation emitted by a device (“physical” source),
a light radiation emitted by a compound (“chemical/biological” source),
   and their combinations.
   Devices Emitting the Light Radiation which Activates Lipase

According to a first embodiment of the invention, the light radiation exhibiting at least one predominant wavelength which activates lipase is emitted by a device.

The said device can be chosen in particular from white light in combination with a specific filter which allows the passage of the said light radiation exhibiting at least one predominant wavelength which activates lipase, lasers, IPL, LEDs and their combinations.

In particular, the said device emits a light radiation exhibiting a predominant wavelength ranging from 400 to 510 nm, preferably from 420 to 500 nm and more preferably from 430 to 470 nm and is chosen from: lasers, IPL, LEDs, a device emitting white light in combination with a specific filter which lets pass through at least one predominant wavelength ranging from 400 to 510 nm (emission spectrum of blue light), and their combinations.

Mention may in particular be made, as devices emitting a light radiation which activates lipase which can be used according to the invention, of:

white light, natural or artificial, in combination with a specific filter or a screening device which lets pass through at least one predominant wavelength which activates lipase (for example wavelength of the emission spectrum of blue light): mention may in particular be made of arc lamps (for example xenon lamp) or incandescent lamps as examples of devices emitting artificial white light. The term “natural white light” is understood to mean daylight, which will be combined with a screening device which specifically lets pass through predominantly a colour (for example blue) which activates lipase.

lasers (for light amplification by the stimulated emission of radiation); this is a light source of very high intensity which is monochromatic. Unlike white light (photons of various wavelengths emitted randomly at different times and in different directions), the light radiation emitted by a laser is a light composed of photons emitted at the same time and in the same direction.

Three elements characterize lasers: the wavelength (λ); the mode of emission: continuous (constant power delivered), pulsed (energy is delivered in pulses, the frequency and the power of which can be adjusted) and ultrapulsed (the pulses have a set duration and power but the power is considerable and the duration is extremely short); and the power: from a few mW to tens of thousands of watts.

IPL: Intense Pulsed Light

The fundamental difference between laser and IPL lies in the fact that IPL can deliver hundreds, indeed even thousands, of colours simultaneously, whereas the laser delivers only a single wavelength. These machines make it possible to choose the wavelength suited to the problem to be treated just by changing the filter. They are also referred to as “noncoherent light sources”. Emission of light pulses of high overall intensity is concerned.
This technology can deliver a broad spectrum of wavelengths which are absorbed by multiple chromophores. Large surface areas can be treated simultaneously.

LEDs: Light-emitting diodes (photomodulation (LED, light-emitting diode, see “The newest medical breakthrough for skin renewal and shrinking pores (2004)”).

LED: the LED generally emits low-intensity light of a few milliwatts; they are classified within the category of low-power lasers (power of 1 to a few tens of mW).

According to a preferred form, use will be made of devices of LED type.

Mention may in particular be made, as examples of devices of LED type which are available commercially, of:
a) devices which can be used in institutes for caring for the skin and/or hair, such as:
   a. Omnilux™ system (415 nm) from Photo Therapeutics Ltd.;
   b. Lumiphase™ system (405 nm) from Opusmed Inc.;
   c. Delphia™ and HydroFacial™ systems (420 nm) from Edge Systems Corp.;
   d. RevitaLight system (420 nm) from Skincare Systems Inc.
b) devices capable of being used at home, such as those sold in particular on the Internet (specialist sites or sales sites, such as e-Bay), such as Lumiderm or Facial Sensation from Photo Rejuvenation.

The light radiation according to the invention is preferably substantially monochromatic (predominant wavelength) with a predominant wavelength ranging from 400 to 510 nm, preferably from 420 to 500 nm and more preferably from 430 to 470 nm, with an optimum peak in the vicinity of 450 nm.

Compounds Emitting a Light Radiation which Activates Lipase

According to another embodiment of the invention or to supplement a device as described above, the light radiation exhibiting at least one predominant wavelength which activates lipase is emitted by a compound chosen from metabolites or active principles emitting, in particular under exposure to light (visible, UV), such a predominant wavelength which activates lipase, a compound which filters the light in order to specifically allows the passage of the said predominant wavelength which activates lipase, and their mixtures.

According to a preferred form, the said compound emits and/or filters a light radiation exhibiting at least one predominant wavelength which activates lipase ranging from 400 to 510 nm, preferably from 420 to 500 nm and more preferably from 430 to 470 nm.

According to a specific form, the said compound emits the said light radiation when it is under exposure to light (visible, UV). This exposure to light can be an exposure to daylight (“natural” light) or an exposure to an artificial light employing a device. In particular, use will be made of a device emitting a white or coloured or UV light intended to excite the said compound in order to allow it to emit the said predominant wavelength (for example blue).

The intensity of this exposure to light will be such that it makes it possible for the said compound to emit the said radiation which activates lipase, in particular at a dose ranging from 0.01 to 200 J/cm², preferably from 0.1 to 30 J/cm², more preferably from 1 to 30 J/cm², indeed even from 5 to 30 J/cm².

These chromic phenomena and the corresponding compounds are described in particular in the reference work “Chromic Phenomena, Technological Applications of Colour Chemistry, Peter Bamfield, The Royal Society of Chemistry, 2001”.

Metabolites and Active Principles which Emit and/or Filter Blue Light

These substances are more particularly metabolites or active principles which emit, in particular under exposure to light (visible, UV), a light radiation exhibiting at least one predominant wavelength ranging from 400 to 510 nm, preferably from 420 to 500 nm and more preferably from 430 to 470 nm, with an optimum peak in the vicinity of 450 nm.

Mention may in particular be made of:

blue-coloured anthocyanins;

blue pigments or dyes;

phosphorescent substances;

fluorescent substances;

and their mixtures.

Mention may in particular be made, by way of examples, of blue-coloured anthocyanins, such as 3-caffeoylquinic acid, esculin, esculetin in an alkaline medium, and their mixtures.

Mention may in particular be made, as blue pigments or dyes, of indigotin, brilliant blue FCF, tetrazolium blue, spirulina blue, azulene, guaiazulene, copper derivatives, such as copper gluconate or chlorophyllin copper, and their mixtures.

Mention may in particular be made, as phosphorescent substances which can be excited under visible or UV light, of phosphorescent pigments.

The term “blue phosphorescent pigment” is understood to mean the conventional phosphorescent pigments listed below but also any cosmetically acceptable substance which emits a phosphorescent blue light with a wavelength of between 400 and 510 nm, preferably from 420 to 500 nm and more preferably from 430 to 470 nm, with an optimum peak in the vicinity of 450 nm, in particular under exposure to light (visible, UV).
The presence of phosphorescent pigments provides the composition with a continuous source of blue light, the phosphorescence is activated by exposure to the UV radiation conventionally present in daylight and its effect lasts several hours.
Mention may in particular be made, as example, of aequorin (coelentrazine) in combination with calcium.

Mention may in particular be made, as fluorescent substances, of optical brighteners resulting in a blue fluorescence, such as Tinopal SCBXS from Ciba (disodium 4,4′-distyrylbiphenyldisulphonate), skimmin (glycoside form of umbelliferone), lumichrome and their mixtures.

In particular, the said compound which emits and/or filters the said light radiation exhibiting at least one predominant wavelength which activates lipase ranging from 400 to 510 nm, preferably from 420 to 500 nm and more preferably from 430 to 470 nm is chosen from blue-coloured anthocyanins, such as 3-caffeoylquinic acid, esculin or esculetin in an alkaline medium, blue pigments or dyes, such as indigotin, brilliant blue FCF, tetrazolium blue, spirulina blue, azulene, guaiazulene or copper derivatives, such as copper gluconate or chlorophyllin copper, aequorin (coelentrazine) in combination with calcium, or optical brighteners resulting in a blue fluorescence, such as disodium 4,4′-distyrylbiphenyldisulphonate (Tinopal SCBXS from Ciba), skimmin (glycoside form of umbelliferone), lumichrome and their mixtures.

According to a preferred form, use will be made of a compound chosen from 3-caffeoylquinic acid, brilliant blue FCF, copper derivatives and their mixtures.

According to another preferred form, use will be made of a compound chosen from 3-caffeoylquinic acid, indigotin, tetrazolium blue, spirulina blue, azulene, guaiazulene, aequorin (coelentrazine) in combination with calcium, disodium 4,4′-distyrylbiphenyldisulphonate (Tinopal SCBXS from Ciba), skimmin (glycoside formed of umbelliferone) and their mixtures.

According to an entirely preferred embodiment, use will be made of 3-caffeoylquinic acid.

According to another entirely preferred embodiment, use will be made of brilliant blue FCF.

According to another entirely preferred embodiment, use will be made of copper derivatives.

According to another entirely preferred embodiment, use will be made of indigotin.

According to another entirely preferred embodiment, use will be made of tetrazolium blue.

According to another entirely preferred embodiment, use will be made of spirulina blue.

According to another entirely preferred embodiment, use will be made of azulene.

According to another entirely preferred embodiment, use will be made of guaiazulene.

According to another entirely preferred embodiment, use will be made of aequorin (coelentrazine) in combination with calcium.

According to another entirely preferred embodiment, use will be made of disodium 4,4′-distyrylbiphenyldisulphonate.

According to another entirely preferred embodiment, use will be made of skimmin.

Other Compounds which Filter Blue Light

Use may also be made, alternatively and/or in combination with the compounds emitting a light radiation which activates lipase described above, of compounds capable of filtering the light in order to allows the passage of specifically the light radiation which activates lipase.

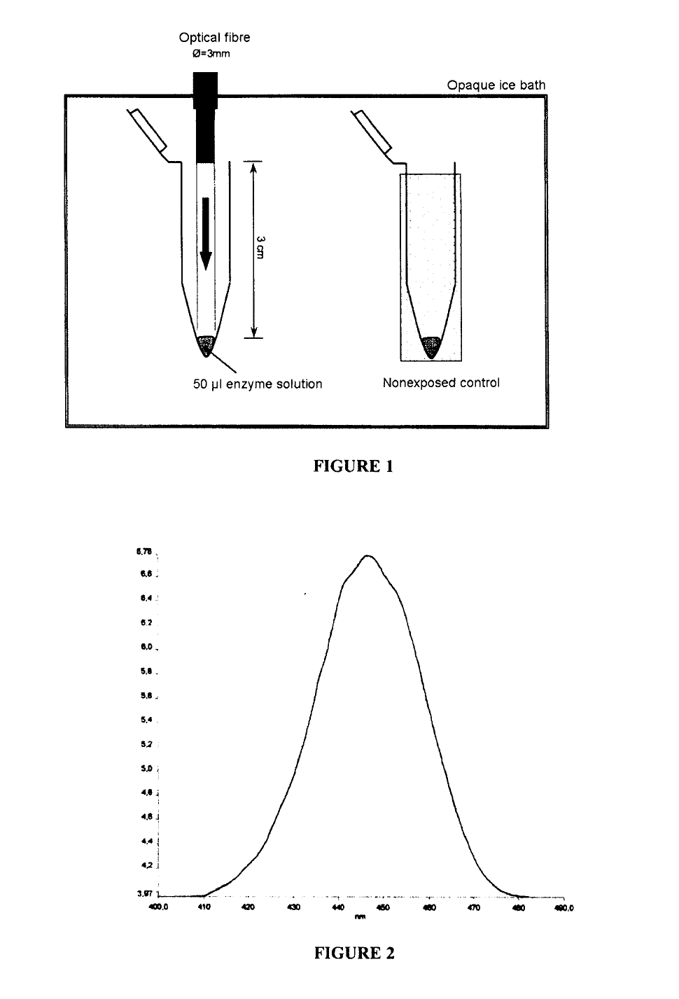

Mention may be made, by way of example, of holographic pigments comprising a polymer matrix in which a volume hologram is recorded, which act as prisms and have the property, on choosing the appropriate refractive index, of emitting a light radiation which activates lipase (for example radiation with a predominant wavelength of 400-510 nm) when they are illuminated with white light.
These systems are described by the Applicant Company in Application FR0758017.
The volume hologram converts the incident light into one or more beams having spatial and spectral dependencies which are a function of the parameters of the hologram.
An example of a volume hologram is the hologram of “Denisyuk” type, which is a reflection hologram which can be obtained in a holographic film using a single laser beam, acting both as object beam and as reference beam, as disclosed in Application EP 1 754 968 A2 and on the site http://www.smartholograms.com/site/sections/technology/creating-sensors.htm, and in Application EP 1 369 681 A1 (see FIGS. 1a and 1b in particular).
These compounds which filter blue light require exposure to natural or artificial light in order to allows the passage of specifically the light radiation which activates lipase.

According to a first form, the said compound which emits and/or filters, in particular under exposure to light, a light radiation exhibiting at least one predominant wavelength which activates lipase is present in the composition comprising the compound bioconvertible by lipase.

According to an alternative form, the said compound which emits and/or filters, in particular under light radiation, a light radiation exhibiting at least one predominant wavelength which activates lipase is present in a composition separate from the composition comprising the compound bioconvertible by lipase.

These compounds which emit and/or filter the light radiation which activates lipase can be used at concentrations sufficient to emit and/or ensure the passage of an amount of light radiation which activates lipase.
The said compound which emits and/or filters, in particular under exposure to light, a light radiation exhibiting at least one predominant wavelength which activates lipase is present in one of the compositions employed in the method in a content ranging from 0.01 to 20% by weight, with respect to the total weight of the said composition, preferably from 0.1 to 10% by weight, with respect to the total weight of the said composition.

These compounds which emit and/or filter the light radiation which activates lipase are used in an amount sufficient to ensure a light emission flux from 0.1 to several tens of mW.

The person skilled in the art will adjust the duration of exposure to this light radiation emitted by the said compound according to characteristics of the said compound and the effect desired.
Purely by way of indication, the areas of skin and/or hair to be treated can receive a total energy (dose) delivered to the skin and/or hair ranging from 0.01 to 200 J/cm², preferably from 0.1 to 30 J/cm², more preferably from 1 to 30 J/cm², indeed even from 5 to 30 J/cm².

A further subject-matter of the invention is a composition comprising, in a physiologically acceptable medium:
a) at least one compound bioconvertible by lipase, and
b) at least one compound which emits and/or filters, in particular under exposure to light, at least one light radiation exhibiting at least one predominant wavelength which activates lipase.

The said compound which emits and/or filters, in particular under exposure to light, a radiation exhibiting at least one predominant wavelength which activates lipase is as defined above.

In particular, it can be chosen from metabolites and active principles which emit and/or filter, in particular under exposure to light, a radiation with a predominant wavelength ranging from 400 to 510 nm, preferably from 420 to 500 nm and more preferably from 430 to 470 nm, a compound which filters light in order to allows the passage of specifically blue light, and their mixtures.

Mention may in particular be made, by way of examples, of blue-coloured anthocyanins, such as 3-caffeoylquinic acid, esculin or esculetin in an alkaline medium, blue pigments or dyes, such as indigotin, brilliant blue FCF, tetrazolium blue, spirulina blue, azulene, guaiazulene or copper derivatives, such as copper gluconate or chlorophyllin copper, aequorin (coelentrazine) in combination with calcium, or optical brighteners resulting in a blue fluorescence, such as disodium 4,4′-distyrylbiphenyldisulphonate, skimmin (glycoside form of umbelliferone), lumichrome and their mixtures.

Preferably, the composition will comprise at least one compound chosen from 3-caffeoylquinic acid, brilliant blue FCF, copper derivatives and their mixtures.

According to a preferred embodiment of the present invention, the composition will comprise 3-caffeoylquinic acid.

According to another preferred embodiment of the present invention, the composition will comprise brilliant blue FCF.

According to another preferred embodiment of the present invention, the composition will comprise copper derivatives.

Examples of compounds bioconvertible by lipase are described above.

Preferably, the compound bioconvertible by lipase is present in the composition in a content ranging from 0.001 to 50% by weight, with respect to the total weight of the said composition, preferably from 0.1 to 30% by weight, with respect to the total weight of the said composition.

Preferably, the compound which emits and/or filters, in particular under exposure to light, a light radiation exhibiting at least one predominant wavelength which activates lipase is present in the composition in a content ranging from 0.01 to 20% by weight, with respect to the total weight of the said composition.

The composition can be a cosmetic or pharmaceutical composition.

Preferably, it will be a cosmetic composition.

According to a first embodiment, the composition is intended for topical application to the skin and/or hair.

According to another embodiment, the composition is intended for oral administration.

According to a preferred form, the composition according to the invention additionally comprises at least one antioxidant, preferably an antiphotooxidant, such as, for example, tocopherols, tocotrienols, β-carotene, baicalin, ferulic acid, phloretin and their mixtures. Preferably, the said composition will comprise at least vitamin E (tocopherol) and its derivatives, preferably tocopherol acetate.

The composition can also comprise an agent which promotes the penetration of the said compounds, such as solvents, desquamating agents and their mixtures.

The person skilled in the art will take care that the choice of these additional ingredients is compatible with the application of a device emitting a light radiation which activates lipase and does not detrimentally affect the desired properties.

A further subject-matter of the invention is a kit comprising:
a) a composition comprising at least one compound bioconvertible by lipase and one compound capable of emitting and/or filtering a light radiation exhibiting at least one predominant wavelength which activates lipase;
b) a first device which makes it possible for the said compound present in the first composition to emit and/or filter the said light radiation exhibiting at least one predominant wavelength which activates lipase.

According to an alternative, the invention also relates to a kit comprising:
a) a first composition comprising at least one compound bioconvertible by lipase;
b) a second composition comprising a compound capable of emitting and/or filtering a light radiation exhibiting at least one predominant wavelength which activates lipase;
c) optionally a first device which makes it possible for the said compound present in the second composition to emit and/or filter the said light radiation exhibiting at least one predominant wavelength which activates lipase.

In the case of a compound capable of emitting the light radiation which activates lipase, the first device excites the said compound so as to cause it to emit the said light radiation which activates lipase.

In particular, the first device can be a device which emits a light radiation exhibiting wavelengths of visible and/or UV light, according to the nature of the compound present in the second composition. The device can emit a light radiation chosen from white light, a coloured light and a UV light. Preferably, it will be visible light and in particular blue light.

In the case of a compound capable of filtering the light radiation which activates lipase, the first device is generally a light radiation which is filtered by the said compound to give a light radiation which activates lipase.

According to a specific form, the kits described above can additionally comprise a second device emitting a light radiation exhibiting at least one predominant wavelength which activates lipase.

The invention also relates to a kit comprising:
a) a composition comprising at least one compound bioconvertible by lipase;
b) a device emitting a light radiation exhibiting at least one predominant wavelength which activates lipase.

The device emitting a light radiation exhibiting at least one predominant wavelength which activates lipase is chosen in particular from a device emitting white light in combination with a specific filter which lets pass through the said light radiation which activates lipase (in particular a light radiation exhibiting at least one predominant wavelength corresponding to the emission spectrum of blue light), lasers, IPL, LEDs and their combinations.

The said compound bioconvertible by lipase is as defined above.

Examples of compounds which emit and/or filter, in particular under exposure to light, a radiation exhibiting at least one predominant wavelength which activates lipase are described above.

According to a preferred form, the constituent composition of the kit or at least one of the constituent compositions of the kit additionally comprises at least one antioxidant, preferably at least one antiphotooxidant.

Examples of such antioxidants, preferably antiphotooxidants, are described above.

The kit according to the invention can be a cosmetic kit or a pharmaceutical kit.

The constituent compositions of these kits can be compositions intended for topical or oral administration.

Preferably, they will be compositions intended for topical administration.

### Formulation

The compositions employed in the method, the composition or the kit according to the invention as described above are intended for topical and/or oral application.

The composition generally comprises a physiologically acceptable medium, that is to say a medium compatible with the skin and/or its superficial body growths. It is preferably a cosmetically acceptable medium, that is to say a medium which exhibits a pleasant smell, a pleasant colour and a pleasant feel and which does not cause unacceptable discomfort (smarting, red blotches, tightness) liable to dissuade the consumer from using this composition.

For oral administration, in particular in an “oral cosmetic”, the composition can be provided in particular in the form of capsules, including hard gelatin capsules, tablets, including sugar-coated tablets, granules, chewing gum, gels, syrups to be taken orally or any other form known to a person skilled in the art.

Preferably, the composition is a topical composition.

The term “topical composition” is understood to mean a composition intended for local application to any surface area of the body, including the skin, the mucous or semi-mucous membranes, the scalp or the hair.

For topical application to the skin, the composition can have the form of an aqueous, aqueous/alcohol or oily solution which is optionally gelled, of an emulsion with a liquid or semi-liquid consistency of the milk type, obtained by dispersing a fatty phase in an aqueous phase (O/W) or vice versa (W/O), of a triple emulsion (W/O/W or O/W/O), of a suspension or emulsion with a soft, semi-solid or solid consistency of cream or gel type, of a liquid, pasty or solid anhydrous product, of microemulsions, of microcapsules, of microparticles, of a vesicular dispersion of ionic type (liposomes or oleosomes) and/or nonionic type (niosomes) and/or of a dispersion of minute spheres.

It is also possible to envisage a composition in the foam form or also in the spray or aerosol form then comprising a pressurized propellant, or also in the patch or impregnated pad form.

The composition can thus be provided in the form of a lotion, serum, milk, O/W or W/O cream, gel, ointment, salve, powder, balm, patch, impregnated pad, soap, bar or foam.

According to the area of the body targeted and the intensity of application desired, a person skilled in the art can choose from different composition forms:

a composition intended to remain applied to the skin even after the exposure to the light radiation which activates lipase; this composition can be a dispersion of the lotion or gel type, an emulsion with a liquid or semi-liquid consistency of the milk type, obtained by dispersing a fatty phase in an aqueous phase (O/W) or vice versa (W/O), a suspension or emulsion with a soft, semi-solid or solid consistency of the cream or gel type, a multiple emulsion (W/O/W or O/W/O), a microemulsion, a vesicular dispersion of ionic and/or nonionic type, or a wax/aqueous phase dispersion;

a composition which remains in contact with the skin only throughout the duration of the exposure to the light radiation which activates lipase, such as a mask, in the form of a cream which the user can specifically apply to the areas to be treated and can then subsequently remove, or a patch impregnated with a compound bioconvertible by lipase; in this administration form, the masks or patches have to be sufficiently transparent to allows the passage of the light radiation which activates lipase.

The topical composition according to the invention comprises an effective amount of compound bioconvertible by lipase preferentially of 0.001 to 50% by weight, preferably of 0.1 to 30% by weight and more preferably still of 1 to 10% by weight, with respect to the total weight of the composition.

According to a particular form, the compositions of the invention can additionally comprise at least one antioxidant, preferably an antiphotooxidant, such as, for example, tocopherols, tocotrienols, β-carotene, baicalin, ferulic acid, phloretin and their mixtures. Use will preferably be made of vitamin E (tocopherol) and its derivatives, preferably tocopherol acetate.

According to a specific form of the invention, it is also possible to sequentially combine an additional oral treatment with a compound active with regard to greasy skin and/or acne, in order to supplement the effects of the combination which is the subject-matter of the invention.

The invention will now be illustrated in the following nonlimiting examples.

FIG. 1: set-up of the in vitro test on the activation of lipase by blue light

FIG. 2: blue light spectrum used in the examples.

### EXAMPLES

### Example 1

### Effect of a Specific Light Radiation on Hydrolysis of Ethyl Lactate

In the presence of lipase, ethyl lactate is hydrolysed to give L-lactate (which reacts with water to give lactic acid) and ethanol. The lactic acid thus formed is quantitatively determined.

The lipase (reference L3126 from Sigma) is exposed with stirring to a light radiation with a predominant wavelength of 400-510 nm (FIG. 2: spectrum of blue light used, intensity 0.5 mW/cm²) in the presence of ethyl lactate (from Sigma or Fluka) according to the set-up described in FIG. 1.

The control corresponds to bringing together the lipase and the ethyl lactate under the same conditions, without exposure to the said light radiation.
An increase in the hydrolysis of the ethyl lactate, with respect to the control, is found. This increase is proportional to the time of exposure of the lipase to the said light radiation, as presented in the following table.

TABLE 1

| | Control | Test | Test - Control |
|---|---|---|---|
| Reaction | (ppm lactic acid | (ppm lactic acid | % of variation |
| time in min | in reaction medium) | in reaction medium) | versus control |
| 20 | 0.45 | 0.48 | 5.16 |
| 40 | 0.62 | 0.69 | 9.08 |
| 90 | 0.69 | 0.84 | 17.94 |

The advantage of this bioconversion also lies in the fact that the ethanol is allowed to be released directly in situ in the deep part of the sebaceous follicle.

Hence the advantage for the formulation because, as ethanol is very volatile, when an emulsion comprising ethanol is applied, the ethanol immediately evaporates during the application by massaging the emulsion on the skin.

This time, the ethyl lactate, due to its lipophilic valency, can thus dissolve in the sebum and go deep, can then be hydrolysed by the lipase and can thus release ethanol and lactic acid active with regard to the P. acnes situated in the depth of the follicle.

### Example 2

### Effect of a Specific Light Radiation on the Hydrolysis of Ascorbyl Palmitate

The same protocol as that described in Example 1 is carried out, with the replacement of ethyl lactate by ascorbyl palmitate.

In the presence of lipase, the ascorbyl palmitate hydrolyses to give palmitic acid and ascorbic acid.
The lipase is exposed with stirring to the said radiation with a predominant wavelength ranging from 400 to 510 nm (blue spectrum) in the presence of ascorbyl palmitate. An increase in the hydrolysis proportional to the time of exposure of the lipase to the said radiation is found, as presented in the following table:

TABLE 2

| | Control | Test | |
|---|---|---|---|
| | (in ppm palmitic | (in ppm palmitic | Test - Control |
| Reaction | acid released in | acid released in | % of variation |
| time in min | reaction medium) | reaction medium) | versus control |
| 20 | 62.89 | 63.96 | 1.71 |
| 40 | 74.87 | 77.15 | 2.95 |
| 60 | 89.16 | 98.99 | 9.92 |
| 90 | 105.5 | 119.51 | 11.72 |

### Example 3

### Examples of Formulations

Unless otherwise indicated in the formulations, the % are shown as % by weight, with respect to the total weight of the composition.

The compositions described below will be applied to the face or to the body immediately before prolonged exposure (30 min to 1 h 30) with a radiation with a wavelength of between 400 and 510 nm.

### Gel for Treating Acne:

| INCI name | % |
|---|---|
| Xanthan gum | 0.4 |
| Potassium hydroxide | 0.3 |
| Dimethicone PEG phosphate | 2 |
| Styrene/maleic acid copolymer, | 1 |
| sodium salt, at 40% by weight in | |
| water (SMA1000HNa ® sold | |
| by Cray Valley) | |
| Cyclohexasiloxane | 10 |
| Ethyl lactate (Puralsolv EL ® | 5 |
| from Purac) | |
| Ethanol | 10 |
| Carbomer | 0.4 |
| Crosslinked acrylic acid/alkyl | 0.25 |
| acrylate polymer (Pemulen | |
| TR2 ® from Lubrizol) | |
| Water | q.s. for 100 |

### Tonic for the Face:

| INCI name | % |
|---|---|
| Triethanolamine | 0.6 |
| Phenoxyethanol | 0.7 |
| Disodium EDTA | 0.2 |
| Styrene/maleic acid copolymer, | 1 |
| sodium salt, at 40% by weight | |
| in water (SMA1000HNa ® sold by | |
| Cray Valley) | |
| Cyclopentasiloxane | 2 |
| Dimethicone | 2 |
| Denatured alcohol | 2 |
| Ethyl salicylate | 0.5 |
| Propylparaben | 0.05 |
| Crosslinked acrylic acid/alkyl | 0.5 |
| acrylate polymer (Pemulen TR2 ® | |
| from Lubrizol) | |
| Tocopherol | 0.1 |
| Brilliant blue FCF (Sensient) | 1 |
| Preservatives | 0.15 |
| Water | q.s. for 100 |

### Care Lotion for Greasy Skin:

| INCI name | % |
|---|---|
| Chlorphenesin | 0.2 |
| Butylene glycol | 4 |
| Phenoxyethanol | 0.5 |
| PEG-32 | 0.5 |
| PEG-8 | 1.5 |
| Styrene/maleic acid copolymer, | 1 |
| sodium salt, at 40% by weight | |
| in water (SMA1000HNa ® sold | |
| by Cray Valley) | |
| Glycerin | 3 |
| Propylene glycol | 0.25 |
| PEG-60 hydrogenated castor oil | 0.17 |
| Tetrasodium EDTA | 0.08 |
| Ascorbyl palmitate (Roche | 2.0 |
| Vitamins) | |
| Preservatives | 0.15 |
| Water | q.s. for 100 |

### Iontophoretic Patch

According to another embodiment, a commercial reference path Ionotopatch™ (Travanti Pharma, Mendota Heights, Minn., USA) is applied to an area treated beforehand with one of the above compositions.

It is subsequently connected to an electric current for delivering a galvanic current generated by difference in potential of 1V and comprising two electrodes, a Zn anode and an AgCl cathode, and is exposed under a source of blue light.

This treatment is carried out at the rate of once daily for 30 to 45 minutes.

### Blue Care Mask

| | |
|---|---|
| Oily phase: | |
| Octyldodecanol | 6% |
| Apricot kernel oil | 6% |
| Triglycerides | 5% |
| Kaolin | 3% |
| Cetyl alcohol | 2% |
| Vitamin E acetate | 0.5% |
| Hydrogenated palm oil | 6% |
| Liquid fraction of shea butter | 5% |
| Aqueous phase: | |
| Xanthan gum | 0.4% |
| Sucrose cocoate/sorbitan stearate | 5.5% |
| (mixture sold by ICL under the | |
| name Arlaton 21121) | |
| Glycerin | 3% |
| Ethanol | 5% |
| Retinol palmitate | 0.2% |
| Brilliant blue FCF | 0.4% |
| Fragrance | 0.3% |
| Preservative | q.s. |
| Water | q.s. for 100 |

This mask is applied to the areas of the skin to be treated and then the subject is placed under a source of white light (natural or electrical) for 45 minutes to 1 hour.

1. A method of improving the appearance of the skin and/or hair comprising administering to the skin and/or hair:
at least one compound bioconvertible by lipase; and
at least one light radiation exhibiting at least one predominant wavelength which activates lipase.

2. The method according to claim 1, wherein the light radiation exhibits at least one predominant wavelength ranging from 400 to 510 nm.

3. The method according to claim 1, wherein the light radiation exhibiting at least one predominant wavelength which activates lipase is applied to the skin and/or hair at a dose ranging from 0.01 to 200 J/cm².

4. The method according to claim 1, wherein the light radiation exhibiting at least one predominant wavelength which activates lipase is emitted by a device selected from the group consisting of a laser, an IPL, a LED, white light in combination with a specific filter which lets pass through said light radiation exhibiting at least one predominant wavelength which activates lipase, and their combinations.

5. The method according to claim 1, wherein the light radiation exhibiting at least one predominant wavelength which activates lipase is emitted by a compound selected from the group consisting of a metabolite emitting a predominant wavelength which activates lipase, an active principle emitting, a predominant wavelength which activates lipase, a compound which filters the light in order to specifically allows the passage of the said predominant wavelength which activates lipase, and their mixtures.

6. The method according to claim 5, wherein the compound emits and/or filters a light radiation exhibiting at least one predominant wavelength which activates lipase ranging from 400 to 510 nm.

7. The method according to claim 6, wherein said compound which emits and/or filters the light radiation exhibiting at least one predominant wavelength which activates lipase ranging from 400 to 510 nm, is selected from the group consisting of a blue-coloured anthocyanin, a blue pigment, a blue dye, guaiazulene, a copper derivative, aequorin in combination with calcium, and an optical brightener resulting in a blue fluorescence.

8. The method according to claim 5, in which said compound which emits and/or filters, a light radiation exhibiting at least one predominant wavelength which activates lipase is present in the composition comprising the compound bioconvertible by lipase.

9. The method according to claim 5, wherein said compound which emits and/or filters, a light radiation exhibiting at least one predominant wavelength which activates lipase is present in a composition separate from the composition comprising the compound bioconvertible by lipase.

10. The method according to claim 5, in which the said compound which emits and/or filters, a light radiation exhibiting at least one predominant wavelength which activates lipase is present in at least one of the compositions employed in the method in a content ranging from 0.01 to 20% by weight, with respect to the total weight of said composition.

11. The method according to claim 1, wherein the compound bioconvertible by lipase is formulated in a composition intended for topical administration to the skin or hair.

12. The method according to claim 1, wherein the compound bioconvertible by lipase is a compound capable of releasing, after hydrolysis by the lipase in situ, at least one agent active with regard to greasy skin having a desquamating, seboregulating and/or antioxidant activity.

13. The method according to claim 1, wherein the compound bioconvertible by lipase is a lipophilic compound.

14. The method according to claim 12, wherein the compound bioconvertible by lipase is a compound comprising one or more ester functional groups possessing a saturated or unsaturated and linear, cyclic or branched chain having from 2 to 50 carbon atoms and optionally comprising one or more substituents.

15. The method according to claim 1, wherein the compound bioconvertible by lipase releases, after hydrolysis in situ by the lipase in the sebaceous follicle, at least one saturated or unsaturated and linear, branched or cyclic compound having from 2 to 50 carbon atoms comprising at least one —COOH group and having a desquamating, seboregulating and/or antioxidant activity.

16. The method according to claim 11, wherein the compound bioconvertible by lipase releases, after hydrolysis in situ by the lipase in the sebaceous follicle, at least one substituted or unsubstituted, mono-, di- or trihydroxylated and linear, cyclic or branched C₂-C₂₂ compound having a desquamating and/or seboregulating and/or antioxidant activity.

17. The method according to claim 1, in which the compound bioconvertible by lipase is an ester selected from the group consisting of an ester of:
α-hydroxy acid, β-hydroxy acid, 10-hydroxy-2-decanoic acid, 4-(2-hydroxyethyl)piperazine-1-ethanesulphonic acid (HEPES), phthalimidoperoxycaproic acid, carboxymethyl-cysteine,
glycerol, ethyl glycerol, pentyleneglycol, caprylyl glycol, resveratrol (C₁₄), retinol (C₂₀), octanol, 2-ethylhexanol, palmityl alcohol, tocopherol, phytosphingosine, octyldodecanol, ascorbic acid, and their mixtures.

18. The method according to claim 1, in which the compound bioconvertible by lipase is at least one selected from the group consisting of ascorbyl palmitate, tocopherol acetate, retinol palmitate and their mixtures.

19. The method according to claim 1, where said compound bioconvertible by lipase is present in the composition in an amount ranging from 0.001 to 50% by weight, with respect to the total weight of the said composition.

20. The method according to claim 1, alleviating visible or tactile irregularities of the surface to the skin and/or scalp.

21. The method according to claim 1, for alleviating the shininess of the skin, alleviating the visibility of the pores, improving the radiance and/or the transparency of the skin, improving the hold of the make-up, improving the texture of the skin and/or preventing and/or reducing the formation of comedones.

22. The method according to claim 1, for preventing and/or combating hair loss, slowing down hair growth, slowing hair greying, reducing the diameter of the hairs and/or the reduction in the vigour of the hairs.

23. A combination of (i) at least one compound bioconvertible by lipase and (ii) of at least one light radiation exhibiting at least one predominant wavelength which activates lipase for regulating the production of sebum and/or killing bacteria.

24. The combination according to claim 23, wherein the compound bioconvertible by lipase is capable of releasing, after hydrolysis in situ by the lipase in the sebaceous follicle, at least one agent active with regard to acne having an antibacterial and/or anti-inflammatory activity.

25. The combination according to claim 23, for preventing and/or reducing red skin blotches of inflammatory origin or preventing and/or treating inflammatory lesions.

26. The combination according to claim 23, for preventing and/or treating the formation of dandruff and/or inflammatory phenomena of the scalp.

27. The combination according to claim 23, wherein the compound bioconvertible by lipase releases, after hydrolysis in situ by the lipase in the sebaceous follicle, at least one saturated or unsaturated and linear, branched or cyclic compound having from 2 to 50 carbon atoms, preferably from 6 to 50 carbon atoms, comprising at least one —COOH group and having an antibacterial and/or anti-inflammatory activity.

28. The combination according to claim 23, wherein the compound bioconvertible by lipase releases, after hydrolysis in situ by the lipase in the sebaceous follicle, at least one substituted or unsubstituted, mono-, di- or trihydroxylated and linear, cyclic or branched C₂-C₂₂ compound having an antibacterial and/or anti-inflammatory activity.

29. The combination according to claim 23, wherein the compound bioconvertible by lipase is selected from the group consisting of esters of:
salicyclic acid, caprylic/capric acid, linoleic acid, benzoic acid, retinoic acid, glycyrrhetinic acid (C₃₀), sorbic acid, asiatic acid (C₄₈), azelaic acid, ursolic acid, pyrrolidonecarboxylic acid, oleanolic acid, betulinic acid, isovaleric acid,
ethanol, 3,7,11-trimethyl-2,6,10-dodecatrienol, oleyl alcohol, chimyl alcohol, batyl alcohol, capryl alcohol, panthenol,
an antibacterial and/or anti-inflammatory, agent
and their mixtures.

30. The combination according to claim 23, wherein the compound bioconvertible by lipase is selected from the group consisting of ethyl salicylate, ethyl lactate, esters of azelaic acid, esters of linoleic acid, esters of glycyrrhetinic acid, and their mixtures.

31. A composition comprising, in a physiologically acceptable medium:
a) at least one compound bioconvertible by lipase, and
b) at least one compound which emits and/or filters at least one light radiation exhibiting at least one predominant wavelength which activates lipase.

32. The composition according to claim 31, wherein said compound which emits and/or filters a radiation exhibiting at least one predominant wavelength which activates lipase is a metabolite or active principle emitting a compound which filters the light in order to specifically allows the passage of said predominant wavelength which activates lipase, and their mixtures.

33. A composition comprising, in a physiologically acceptable medium:
a) at least one compound bioconvertible by lipase according to claim 12, and
b) at least one compound which emits and/or filters at least one light radiation exhibiting at least one predominant wavelength which activates lipase.

34. The composition according to claim 31, wherein the compound bioconvertible by lipase is present in the composition in a content ranging from 0.001 to 50% by weight, with respect to the total weight of the said composition.

35. The composition according to claim 31, wherein the compound which emits and/or filters, a light radiation exhibiting at least one predominant wavelength which activates lipase is present in the composition in a content ranging from 0.01 to 20% by weight, with respect to the total weight of the said composition.

36. The composition according to claim 31, wherein said composition additionally comprises at least one antioxidant.

37. A kit comprising:
a) a composition comprising at least one compound bioconvertible by lipase and one compound capable of emitting and/or filtering a light radiation exhibiting at least one predominant wavelength which activates lipase;
b) a first device which makes it possible for said compound present in the first composition to emit and/or filter the said light radiation exhibiting at least one predominant wavelength which activates lipase.

38. A kit comprising:
a) a first composition comprising at least one compound bioconvertible by lipase;
b) a second composition comprising a compound capable of emitting and/or filtering a light radiation exhibiting at least one predominant wavelength which activates lipase;
c) optionally a first device which makes it possible for the said compound present in the second composition to emit and/or filter the said light radiation exhibiting at least one predominant wavelength which activates lipase.

39. The kit according to claim 37, wherein the first device emits a light radiation selected from the group consisting of white light, a coloured light and a UV light.

40. The kit according to claim 37, wherein said kit additionally comprises a second device emitting a light radiation exhibiting at least one predominant wavelength which activates lipase.

41. A kit comprising:
a) a composition comprising at least one compound bioconvertible by lipase,
b) a device emitting a light radiation exhibiting at least one predominant wavelength which activates lipase.

42. The kit according to claim 41, wherein the device emitting a light radiation exhibiting at least one predominant wavelength which activates lipase is selected from the group consisting of a laser, an IPL, a LED, white light in combination with a specific filter which lets pass through the said light radiation exhibiting at least one predominant wavelength which activates lipase, and their combinations.

43. The kit according to claim 37, wherein the compound bioconvertible by lipase is as a compound capable of releasing, after hydrolysis by the lipase in situ, at least one agent active with regard to greasy skin having a desquamating, seboregulating and/or antioxidant activity.

44. The kit according to claim 37, wherein the compound which emits and/or filters, in particular under exposure to light, a light radiation exhibiting at least one predominant wavelength which activates lipase is a metabolite or active principles emitting a compound which filters the light in order to specifically allow the passage of the said predominant wavelength which activates lipase, and their mixtures.

45. The kit according to claim 37, wherein the constituent composition of the kit or at least one of the constituent compositions of the kit additionally comprises at least one antioxidant.

46. A composition comprising, in a physiologically acceptable medium:
a) at least one compound bioconvertible by lipase according to claim 24, and
b) at least one compound which emits and/or filters at least one light radiation exhibiting at least one predominant wavelength which activates lipase.

47. A composition comprising, in a physiologically acceptable medium:
a) at least one compound bioconvertible by lipase according to claim 27, and
b) at least one compound which emits and/or filters at least one light radiation exhibiting at least one predominant wavelength which activates lipase.

48. The kit according to claim 37, wherein the compound bioconvertible by lipase is a compound capable of releasing, after hydrolysis in situ by the lipase in the sebaceous follicle, at least one agent active with regard to acne having an antibacterial and/or anti-inflammatory activity.

49. The kit according to claim 37, wherein the compound bioconvertible by lipase is a compound which releases, after hydrolysis in situ by the lipase in the sebaceous follicle, at least one saturated or unsaturated and linear, branched or cyclic compound having from 2 to 50 carbon atoms, preferably from 6 to 50 carbon atoms, comprising at least one —COOH group and having an antibacterial and/or anti-inflammatory activity.
